(19) United States
(12) Patent Application Publication
(10) Pub. No.: US 2004/0191296 A1
(43) Pub. Date: Sep. 30, 2004

(54) Food additive

(76) Inventor: Joshua L. Sternberg, Brooklyn, NY (US)

Correspondence Address: OSTROLENK FABER GERB & SOFFEN, 1180 AVENUE OF THE AMERICAS, NEW YORK, NY, 100368403

(21) Appl. No.: 10/610,102

(22) Filed: Jun. 30, 2003

Related U.S. Application Data
(60) Provisional application No. 60/456,886, filed on Mar. 24, 2003.

(51) Int. Cl. A61K 47/00; A61K 33/00

(52) U.S. Cl. 424/439; 424/717

(57) ABSTRACT

A method for adjusting the acidity levels of ordinary food ingested by humans comprises adding to food products potassium bicarbonate in a manner which allows the potassium bicarbonate to be dissolved in the foodstuff and to alter its acidity such that when it is ingested by the human body, the human body is able to maintain in vivo acidity levels that are below those which would have been obtained if the potassium bicarbonate would not have been pre-dissolved in the foodstuff.

### CROSS-REFERENCE TO RELATED APPLICATION

This application is based on and claims priority to U.S. provisional patent Application No. 60/456,886 filed Mar. 24, 2003, the contents of which are incorporated by reference herein.

### BACKGROUND OF THE INVENTION

The present invention is generally directed to food additives and, more particularly, to a food additive that is utilized in a process for promoting bone health.

The prior art contains a body of literature that extols the benefits and medicinal effects of potassium bicarbonate (KHCO₃), and potassium salts of carboxylic acids which alkalinize in vivo, e.g., potassium qluconate (C₆H₅K₃O₇) and potassium citrate (C₆H₁₁KO₇). The use of potassium bicarbonate is particularly recommended for the treatment of osteoporosis. Representative of this prior art are several published articles including:

1. Estimation of the Net Acid Load of the Diet of Ancestral Preagricultural Homo Sapiens and Their Hominid Ancestors, Anthony Sebastian et al., American Journal of Clinical Nutrition, Vol. 76, No. 6, 1308-1316 (December 2002);

2. Improved Mineral Balance and Skeletal Metabolism and Post Menopausal Women Treated With Potassium Bicarbonate, Anthony Sebastian et al., The New England Journal of Medicine, Vol. 330, No. 25, 1776-1781 (Jun. 23, 1994);

3. Neutralization of Western Diet Inhibits Bone Resorption Independently of K Intake and Reduces Cortisol Secretion in Humans, Marc Maurer, American Journal Renal Physiology, Vol. 284, Issue 1, F32-F40, 203 (January 2003), First published Sep. 24, 2002; and

4. Potassium Bicarbonate Reduces Urinary Nitrogen Excretion in Postmenopausal Women, L. Frassetto, et al., The Journal of Clinical Endocrinology & Metabolism, Vol. 82, No. 1, 254-259 (1997).

Indeed, U.S. Pat. No. 5,171,583 explicates the virtues and benefits of potassium bicarbonate as a medicinal treatment of osteoporosis. The contents of U.S. Pat. No. 5,171,583 are incorporated by reference herein. Also incorporated by reference are the teachings of U.S. Pat. Nos. 5,445,805; 5,496,569; 5674,529; 5,766,640; and 6,027,737. These incorporated by reference patents further extol the virtues of providing to the human body potassium bicarbonate.

In general, the prior art, including the prior art referenced above, proposes and describes the use of potassium bicarbonate as a medicament or a pharmacologically prescribed medicine that is taken separately in the form of a regimented medicine. Many individuals do not have the discipline or are generally disinclined to take medicine in the form of pills or injections or even powder inhalation or absorptions or even powder dissolved in water, as long as it is viewed as a medicinally indicated ingredient.

### SUMMARY OF THE INVENTION

The general objective of the present invention is to promote the ingestion of potassium bicarbonate in a manner and form that removes its stigma as a medicine.

The foregoing and other object of the invention are realized by the method of the present invention which discloses the use of a food grade potassium of bicarbonate in powder form as a food and nutritional supplement by humans,—men and women—to be ingested daily, not separately, but rather always in conjunction with the intake of other foods in which it is hidden by being sprinkled thereon. Preferably the daily amount to be taken is about 2 teaspoons.

The foregoing and other object of the invention are described more fully below.

### DETAILED DESCRIPTION OF PREFERRED EMBODIMENTS OF THE INVENTION

In marked departure from the prior art cited above, the present invention is directed to the method of ingesting food as well as to the preparation of foodstuff normally consumed by individuals in which the potassium bicarbonate (or equivalent elements) are dissolved into the food prior to its ingestion, i.e., in vitro. The key is that the method and food are effective to neutralize the food acid content and alter its alkalinity so that the methods and approach of the prior art which operate in vivo are substantially avoided.

Primarily, the present invention distinguishes itself from the prior art in the fact that it relates to an in vitro process rather than to the in vivo process of the prior art. Whereas the prior art aims at introducing into the body potassium bicarbonate (or equivalents) for the purpose of re-balancing the acidity/alkalinity of the human in vivo chemistry, the present invention seeks to avoid the re-balancing step altogether (assuming that it is even effective) by altering the diet that is ingested so that the problem does not arise in the first instance. The potassium bicarbonate additive of the present invention does not remain intact but rather is dissolved with the foodstuff in a manner which should be effective to neutralize the hydrogen ions that are present in the food products.

The present invention utilizes a food grade potassium bicarbonate in powder form as a food and nutritional supplement that is to be consumed by humans—men and women—by being ingested daily in the amount of approximately 2 teaspoons. This potassium bicarbonate powder has been found to be effective when sprinkled on a person's daily food intake (salads, vegetables, meat and fish) as a condiment which will give it a slightly salty taste or dissolved in daily beverage intake such as coffee, tea, milk, juice and soup. The diet may be liberal and not limited to a constant amount of calcium, phosphorous, sodium, potassium or protein. Potassium bicarbonate is a safe and non-toxic material. LD-50 in rats is approximately 2825 mg/kg of body weight which would be about 169 grams/60 kg of human weight. The molecular weight of potassium bicarbonate is 100.12 grams whereby a millimole equals 0.1 grams. The density of potassium bicarbonate is 68 lb/cu. ft. Using appropriate conversions, 2 teaspoons is equivalent to about 109 millimoles. This is the range of a daily amount of 60 to 120 millimoles per 60 kg of body weight, but the method of ingestion is not limited to aqueous solution.

Although the other diet variables are not constant, the potassium bicarbonate as shown in the literature cited will decrease the body's acid load, which would otherwise prevail due to the ordinary western diet consumed by individuals and which is responsible for decreased bone formation and increased bone resorption. The preparation of food in accordance with the present invention and the ingestion thereof is substantially different from the methodology described in the referenced prior art where the potassium bicarbonate is taken as a medicinal element, separate and apart from the food and where it is intended to be ingested as potassium bicarbonate into the body in order to ameliorate and/or prevent already occurring acid imbalances.

By assuring that the body's acid production is neutralized and is not allowed to unduly increase, the bone preservation is accomplished. In accordance with the present invention, the utilization of potassium bicarbonate as a nutritional supplement is carried out in and as part of the convenient activities of daily living, in a user friendly setting inasmuch as the mechanism of its dissolving in the food and its ingestion is carried out in the course of normal eating habits of the individual.

With the present invention, the body's normal acid reduction which suppresses bone formation and stimulates resorption of bone is avoided. By ingestion of this food supplement, particularly in all adult men and women, and especially postmenopausal women who are prone to osteoporosis, strong and healthy bones are maintained. That is, scientific evidence for this use is contained in the referenced prior art.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A method for preparing food for ingesting by humans to maintain optimized body acid levels, comprising:
admixing a pharmacologically acceptable alkalizing potassium salt capable of reducing the acidity of foodstuff on food products that is to be ingested, in a manner which dissolves the pharmacologically acceptable alkalizing potassium salt into the foodstuff and reduces its acidity; and
ingesting the foodstuff with the dissolved pharmacologically acceptable alkalizing potassium salt in an amount and frequency that is effective to maintain body acidity levels that are lower than those which would have been obtained if the food was ingested without the pharmacologically alkalizing potassium salt dissolved therein.

2. The method of claim 1, wherein the pharmacologically acceptable alkalizing potassium salt is potassium bicarbonate.

3. The method of claim 2, wherein the quantity of potassium bicarbonate mixed into the foodstuff to be ingested in an amount of about 60 to 120 millimoles per 60 kg of body weight per 24 hour period.

4. A non-liquid human foodstuff that promotes bone strength in humans, comprising: foodstuff substantially in solid form by reference to its volume and weight and the foodstuff including sufficient liquid content that is able to have dissolved in it potassium bicarbonate in an amount that delivers to a human approximately 60-120 millimoles of potassium bicarbonate per 60 kg body weight over the course of a 24 hour period.
